(19) United States
(12) Patent Application Publication
(10) Pub. No.: US 2021/0291073 A1
(43) Pub. Date: Sep. 23, 2021

(54) COLD EXTRACTION METHOD FOR CANNABINOIDS AND TERPENES FROM CANNABIS BY POLYUNSATURATED LIPID-BASED SOLVENTS

(71) Applicant: Neptune Wellness Solutions Inc., Laval (CA)

(72) Inventors: Fereshteh FAROKHI, Sherbrooke (CA); Pierre ST-JEAN, Laval (CA); Etienne VILLENEUVE, Laval (CA)

(21) Appl. No.: 17/266,425

(22) PCT Filed: Aug. 8, 2019
(86) PCT No.: PCT/CA2019/051089

§ 371 Date: Feb. 5, 2021

Related U.S. Application Data
(60) Provisional application No. 62/716,189, filed on Aug. 8, 2018.

(51) Int. Cl. B01D 11/02

(52) U.S. Cl. CPC B01D 11/0288; B01D 11/0292; A61K 36/73; B01D 11/0265; B01D 11/028

(57) ABSTRACT

The invention relates to methods for producing botanical extracts comprising cannabinoids and terpenes using cold extraction with highly polyunsaturated lipid solvents. These methods allow for the extraction of cannabinoids and terpenes while leaving behind impurities that are commonly found in organic solvent extraction methods.

### FIELD OF THE INVENTION

The present invention relates generally to a process for high recovery of cannabinoids, terpenes and other bioactive molecules from plants of Cannabis genus without the use of organic solvents.

### BACKGROUND OF THE INVENTION

Cannabis has been traditionally used medicinally, especially as a mild analgesic and tranquillizer, but different conventional agents have replaced its use, and controlled prescribing discontinued.

Recently, Cannabis has been shown to have valuable anti-emetic properties that help to reduce the side-effects of nausea and vomiting caused by cancer chemotherapeutic agents. Cannabis also has been shown to possess properties that may be of value in other medical conditions. There is now scientific evidence that Cannabis may give relief to patients suffering from chronic pain, multiple sclerosis, glaucoma, asthma, migraine, epilepsy, and other conditions. The non-intoxicating cannabinoid, cannabidiol (CBD), has been shown to have anti-inflammatory properties that are potentially useful in the treatment of symptoms of arthritis.

A need exists for a method of extracting cannabinoids, terpenes and other bioactive compounds from Cannabis to resolve the problems of other prior techniques, such as efficiency, selectivity and the presence of unwanted contaminants in the final product. The present invention solves these problems.

### SUMMARY OF THE INVENTION

The disclosure provides methods of preparing a botanical extract and the extracts that are obtained therefrom.

The disclosure provides methods of preparing botanical extracts comprising: (a) providing a plant material; (b) contacting the plant material with a lipid solvent, (c) extracting at least one bioactive molecule from the plant material into the lipid solvent for a period of time, and (d) recovering the lipid solvent comprising the botanical extract.

In some embodiments of the methods of the disclosure, contacting the lipid solvent with the plant material comprises releasing the lipid solvent from a solvent chamber into the extraction chamber.

The disclosure provides methods of preparing botanical extracts comprising (a) providing a plant material in an extraction chamber; (b) releasing a lipid solvent from a solvent chamber into the extraction chamber; (c) extracting at least one bioactive molecule from the plant material into the lipid solvent for a period of time; (d) draining the lipid solvent from the extraction chamber into a cold filtration/centrifugation system; and (e) recovering the lipid solvent from the filtration/centrifugation system thereby producing a botanical extract.

The disclosure provides methods of preparing botanical extracts comprising: (a) providing a plant material in an extraction chamber; (b) releasing a lipid solvent from a solvent chamber into the extraction chamber; (c) extracting at least one bioactive molecule from the plant material into the lipid solvent for a period of time thereby producing a lipid solvent comprising a botanical extract; (d) filtering the lipid solvent comprising the botanical extract using a cold filtration/centrifugation system; and (e) recovering the lipid solvent from the filtration/centrifugation system thereby producing a botanical extract.

In some embodiments of the methods of the disclosure, the plant material is heated prior to placing it into the extraction chamber. In some embodiments, the plant material is heated prior to step (a). In some embodiments, the plant material is heated to a temperature of about between 115° C. to 145° C. In some embodiments, the plant material is heated to a temperature of about between 110° C. to 145° C.

In some embodiments of the methods of the disclosure, the period of time is no more than 1 hour. For example, the period of time is 15, 20, 30, or 50 minutes. In some embodiments, the period of time is between about 30 and about 60 minutes. In some embodiments, the period of time is between about 10 and about 30 minutes.

In some embodiments of the methods of the disclosure, the methods further include agitating the contents of the extraction chamber during step (c). For example, the contents are agitated during all or part of the period of time.

In some embodiments of the methods of the disclosure, the methods comprise sonicating the Cannabis plant material and the lipid solvent prior to step (d). The sonication can occur during all of step (c) or part of the first period of time, or for a specified second period of time. In some aspects the first and the second period of time is the same. In some embodiments, the sonication occurs during step (c). In some embodiments, the sonication occurs before step (c).

In some embodiments of the methods of the disclosure, the lipid solvent is at a temperature of about between 0° C. to −40° C. In some embodiments, step (c) is at about −5° C. to about −20° C., about −5° C. to about −10° C., or about 5° C. to about −15° C.

In some embodiments of the methods of the disclosure, the lipid solvent comprises polyunsaturated fatty acids (PUFA). In some embodiments, the PUFA comprise omega-3 fatty acids. In some embodiments, the lipid solvent has a melting temperature below 0° C. In some embodiments, the lipid solvent has a melting temperature between about −8° C. to about −40° C. In some embodiments, the lipid solvent is selected from the group consisting of fish oil, flax seed oil, camelina oil, evening primrose oil, black current oil, ahiflower seed oil, and a combination thereof

In some embodiments of the methods of the disclosure, the recovered lipid solvent from step (e) is returned to the extraction chamber and steps (b) through (e) are repeated. In some embodiments, steps (b) through (e) are repeated 2×, 3×, 4×, 5×, 6×, 7×, 8×, 9×, or 10×. In some embodiments, un-extracted plant material is added to the extraction chamber with the lipid extract.

In some embodiments of the methods of the disclosure, the botanical extract is bleached.

In some embodiments of the methods of the disclosure, the botanical extract is subject to one or more additional purification methods. In some embodiments, the one or more additional purification methods comprise molecular distillation or high-performance liquid chromatography (HPLC).

In some embodiments of the methods of the disclosure, the plant material is fresh or dried. In some embodiments, the plant material is intact or milled. In some embodiments, the plant material is Cannabis. In some embodiments, the Cannabis is Cannabis sativa, Cannabis indica or Cannabis ruderalis. In some embodiments, the plant material is a hybrid. In some embodiments, the Cannabis is industrial hemp.

In some embodiments of the methods of the disclosure, the methods comprise winterizing.

In some embodiments of the methods of the disclosure, the methods comprise de-waxing.

In some embodiments of the methods of the disclosure, the at least one bioactive molecule comprises a cannabinoid, a flavonoid or a terpene. In some embodiments, the cannabinoid comprises Δ⁹-tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof. In some embodiments, the cannabinoid comprises a combination of THC and CBD. In some embodiments, the cannabinoid comprises a combination of THC, THCA, CBD and CBDA. In some embodiments, the terpene comprises myrcene, terpinolene, β-caryophyllene, selina-3 7(11)-diene, guaiol, 10-epi-y-eudesmol, β-eudesmol, α-eudesmol, bulnesol, α-bisabolol or a combination thereof.

The disclosure provides a botanical extract produced by the methods of the disclosure.

In some embodiments of the botanical extracts of the disclosure, the botanical extract is a resin. In some embodiments, the botanical extract is a liquid. In some embodiments, the liquid comprises a lipid solvent, for example a lipid solvent elected from the group consisting of fish oil, flax seed oil, camelina oil, evening primrose oil, black current oil, ahiflower seed oil, and a combination thereof.

The disclosure provides a botanical extract comprising at least one cannabinoid and a lipid solvent. In some embodiments, the lipid solvent is selected from the group consisting of fish oil, flax seed oil, camelina oil, evening primrose oil, black current oil, ahiflower seed oil, and a combination thereof. In some embodiments, the botanical extract comprises at least one terpene. In some embodiments, the botanical extract comprises at least one flavonoid.

The disclosure provides compositions comprising the botanical extracts of the disclosure and a pharmaceutically acceptable carrier, diluent or excipient.

In some embodiments of the compositions of the disclosure, the composition is formulated for oral administration. In some embodiments, the composition is formulated as a liquid, gel, softgel, powder, tablet, caplet, capsule, gelcap, food additive, drop, beverage, pill, lozenge, rinse, paste or gum.

In some embodiments of the compositions of the disclosure, the composition is formulated for topical administration. In some embodiments, the composition is formulated as a liquid, gel, cream, ointment, lotion, salve, balm or paste.

In some embodiments of the compositions of the disclosure, the composition is formulated for transmucosal administration, parenteral administration, subdermal administration, or inhalation. In some embodiments, the transmucosal administration comprises buccal administration or intra-nasal administration.

The disclosure provides methods of making Cannabis extract compositions, comprising: (a) providing a botanical extract produced using the methods of the disclosure, and (b) mixing the botanical extract with a pharmaceutically acceptable carrier, diluent or excipient.

In some embodiments of the methods of making Cannabis extract compositions of the disclosure, the composition is formulated for oral administration. In some embodiments, the composition is formulated as a liquid, gel, softgel, powder, tablet, caplet, capsule, gelcap, food additive, drop, beverage, pill, lozenge, rinse, paste or gum.

In some embodiments of the methods of making Cannabis extract compositions of the disclosure, the composition is formulated for topical administration. In some embodiments, the composition is formulated as a liquid, gel, cream, ointment, lotion, salve, balm or paste.

In some embodiments of the methods of making Cannabis extract compositions of the disclosure, the composition is formulated for transmucosal administration, parenteral administration, subdermal administration, or inhalation. In some embodiments, the transmucosal administration comprises buccal administration or intra-nasal administration.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIG. 1 is a flow diagram of a lipid solvent cannabinoid and terpene extraction procedure in accordance with the present invention.

FIG. 2 shows the relative efficiency for THC, CBD, total cannabinoids and terpenes from a “polyunsaturated fatty acid (PUFA)-rich” oil extraction method at room temperature (RT) and −15° C. after 10 or 30 minutes of extraction. Extract results (%) are compared to control extraction with methanol/chloroform (9:1).

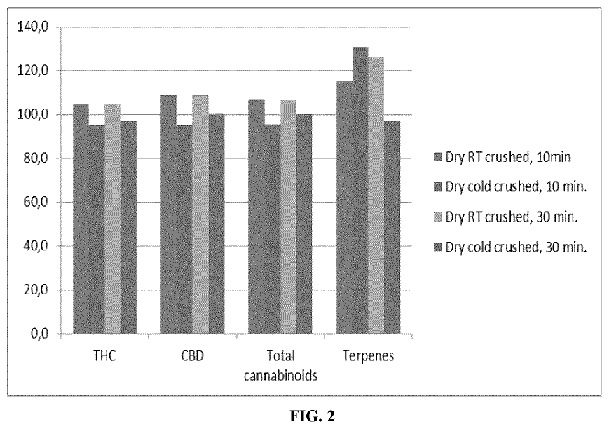

FIG. 3 shows the relative efficiency for terpenes of “PUFA-rich” oil extraction method at RT and −15° C. after 10 or 30 minutes of extraction. Extract results (%) are compared to control extraction with methanol/chloroform (9:1).

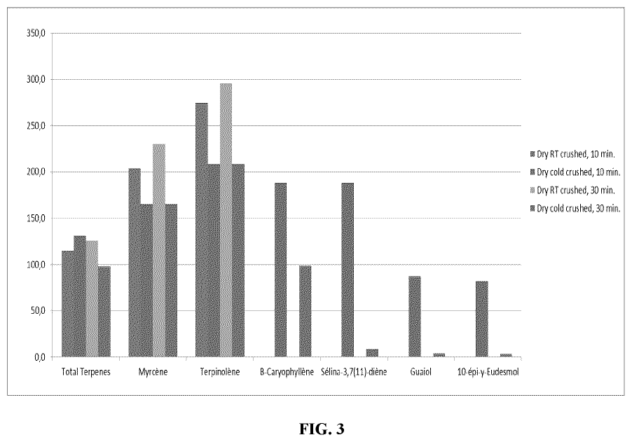

FIG. 4 shows the extraction level of chlorophyll from a “PUFA-rich” oil extraction at −15° C. after 10 or 30 minutes of extraction. The control extraction was done with methanol/chloroform (9:1) at room temperature (RT). The chlorophyll extraction was done on dry cold Cannabis material by ahiflower and camelina seed oil extraction. Results are expressed in ppm (mg/kg dry Cannabis). Control extractions correspond to methanol/chloroform (9:1) extraction.

### DETAILED DESCRIPTION OF THE INVENTION

The invention relates to an extraction process of bioactive compounds from plant material. More specifically, the invention provides methods for extracting and isolating compounds such as pure cannabinoids, cannabinoid acids, terpenes, terpenoids, flavonoids or other bioactive molecules from Cannabis plant material at low temperature by using polyunsaturated lipid solvents that have a melting point below 0° C.

In particular, the extraction methods of the instant disclosure maximize extraction efficiency and minimize contaminants, such as organic solvents, and impurities such as waxes and chlorophyll. By minimizing contaminants and using lipid solvents safe for human consumption, the extraction product is safe and non-toxic.

Cannabis

Cannabis is a genus of plants that include three species, Cannabis sativa, Cannabis indica, and Cannabis ruderalis. More generally, Cannabis also is categorized as either marijuana or hemp based on the natural amount of Δ⁹-tetrahydrocannabinol (THC) present in the plant material, with marijuana being high in THC and hemp having negligible to no amount of THC. This genus has long been used for its hemp fiber material, as well as milk, seeds and oils, for medicinal purposes, and for recreational use. Cannabis species contain a highly complex mixture of compounds, and up to 568 unique molecules have been identified to date (Lewis, M. M. et al., Chemical Profiling of Medical Cannabis Extracts, ACS Omega (2017) 2(9): 6091-6103), any one of which are potentially bioactive in humans. Exemplary bioactive molecules in Cannabis comprise cannabinoids, terpenes and flavonoids.

A variety of strains and hybrids of Cannabis will be known to the person of ordinary skill in the art, all of which can be used as starting material to produce botanical extracts using the methods of the instant invention. Different Cannabis strains produce different amounts of various cannabinoids and/or terpenes, and choice of Cannabis strain(s) or hybrid(s) can contribute to the cannabinoid and/or terpene composition of the botanical extracts produced using the methods described herein. The person of ordinary skill in the art will be able to select the starting Cannabis strain or hybrid most suited to the desired cannabinoid and/or terpene composition of the resulting botanical extract. For example, high cannabidiol (CBD) strains include Charlotte's Web, Cannatonic, AC/DC, Harlequin, Ringo's Gift, Harle-Tsu, Nebula and Sour Tsunami. Exemplary high Δ⁹-tetrahydrocannabinol (THC) strains include Girl Scout Cookies (GSC), Kosher Kush, Ghost OG, Bruce Banner, Ghost Train Haze, Chemdawg, Ace of Spades, Afghani, Afgoo, AK-47, Alien OG, Alien Rock Candy, Allen Wrench, Animal Cookies, Sour Diesel, Skywalker, GG4, The White, Death Star, White Fire OG, Kimbo Kush, Headband, Cherry Pie, Bubba Kush, SFV OG, LA Confidential and Triangle Kush. An exemplary high tetrahydrocannabivarin (THCV) strain includes Dutch Treat.

Any part of the Cannabis plant may be used in the extraction methods of the instant disclosure. For example, stems, leaves, seeds, flowers or a combination thereof can be used as the starting material for the extraction methods of the invention. In some aspects, one or more parts of the plant are used in practicing the claimed methods. Alternatively, all parts of the plants may be used in practicing the claimed methods.

### Cannabinoids

In some embodiments, the instant disclosure provides methods of producing botanical extracts comprising cannabinoids, and compositions comprising botanical extracts comprising cannabinoids.

Cannabinoids are a class of chemical compounds that act on the cannabinoid receptors, also known as the endocannabinoid system in cells. Cannabinoids include endocannabinoids, produced naturally in the body by animals; phytocannabinoids, produced by Cannabis and other plants; and synthetic cannabinoids, which are manufactured. Phytocannabinoids, sometimes also referred to herein as cannabinoids, are a structurally diverse class of molecules that are derived from a common C21 precursor (cannabigerolic acid, or CBGA) or its C19 analog (cannabigerovaric acid, or CBGVA).

There are currently over 100 cannabinoids known to be produced by Cannabis plants, all of which can be purified using the methods of the instant disclosure. Cannabinoids are described in, for example, Brenneisen R. (2007) Chemistry and Analysis of Phytocannabinoids and Other Cannabis Constituents. In: ElSohly M. A. (eds) Marijuana and the Cannabinoids. Forensic Science and Medicine; Humana Press; pp. 17-49. Exemplary cannabinoids include Cannabichromenes such as Cannabichromene (CBC), Cannabichromenic acid (CBCA), Cannabichromevarin (CBCV) and Cannabichromevarinic acid (CBCVA); Cannabicyclols such as Cannabicyclol (CBL), Cannabicyclolic acid (CBLA) and Cannabicyclovarin (CBLV); Cannabidiols such as Cannabidiol (CBD), Cannabidiol monomethylether (CBDM), Cannabidiolic acid (CBDA), Cannabidiorcol (CBD-C1), Cannabidivarin (CBDV) and Cannabidivarinic acid (CBDVA); Cannabielsoins such as Cannabielsoic acid B (CBEA-B), Cannabielsoin (CBE) and Cannabielsoin acid A (CBEA-A); Cannabigerols such as Cannabigerol (CBG), Cannabigerol monomethylether (CBGM), Cannabigerolic acid (CBGA), Cannabigerolic acid monomethylether (CBGAM), Cannabigerovarin (CBGV) and Cannabigerovarinic acid (CBGVA); Cannabinols and cannabinodiols such as Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1) and Cannabivarin (CBV); Cannabitriols such as 10-Ethoxy-9-hydroxy-delta-6a-tetrahydrocannabinol, 8,9-Dihydroxy-delta-6a-tetrahydrocannabinol, Cannabitriol (CBT) and Cannabitriolvarin (CBTV); Delta-8-tetrahydrocannabinols such as Delta-8-tetrahydrocannabinol (Δ⁸-THC) and Delta-8-tetrahydrocannabinolic acid (Δ⁸-THCA); Delta-9-tetrahydrocannabinols such as Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV) and Delta-9-tetrahydrocannabivarinic acid (THCVA); as well as 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC) and 3,4,5,6-Tetrahydro-7-hydroxy-alpha-alpha-2-trimethyl-9-n-propyl-2,6-methano-2H-1-benzoxocin-5-methanol (OH-iso-HI-ICV).

The principle cannabinoid components present in plants of the Cannabis species are the cannabinoid acids, Δ⁹-tetrahydrocannabinolic acid (Δ⁹-THCA or THCA) and cannabidiolic acid (CBDA), with small amounts of the corresponding neutral cannabinoids, respectively, i.e., Δ⁹-tetrahydrocannabinol (Δ⁹-THC or THC) and cannabidiol (CBD). Other cannabinoid acids include CBGA (cannabigerolic acid), CBCA (cannabichromenenic acid), CBGVA (cannabigerovarinic acid), THCVA (tetrahydrocanabivarinic acid), CBDVA (cannabidivarinic acid), CBCVA (cannabichromevarinic acid).

Other neutral cannabinoids include CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethylether), CBE (cannabielsoin), and CBT (cannabicitran).

### Terpenes

In some embodiments, the instant disclosure provides methods of producing a botanical extract comprising terpenes. In some embodiments, the botanical extract comprises terpenes and cannabinoids. In some embodiments, the botanical extract comprises terpenes, cannabinoids and flavonoids.

Terpenes, sometimes referred to as terpenoids, are essential oil (EO) components present in numerous botanicals, including Cannabis, and form the largest group of plant chemicals, with 15-20,000 terpenes that have been fully characterized (Langenheim J H. Higher plant terpenoids: A phytocentric overview of their ecological roles. J Chem Ecol. 1994 June; 20(6):1223-80). Terpenes comprise a large group of compounds synthesized from C₁₀ isoprene subunits. The European pharmacopoeia, Sixth Edition (2007), lists 28 EOs (Pauli A, Schilcher H (2010). In vitroantimicrobial activities of essential oils monographed in the European Pharmacopoeia 6th Edition. In: Baser K H C, Buchbauer G (eds). Handbook of Essential Oils: Science, Technology, and Applications. CRC Press: Boca Raton, Fla., pp. 353-548). Terpenoids are pharmacologically versatile: they are lipophilic, interact with cell membranes, neuronal and muscle ion channels, neurotransmitter receptors, G-protein coupled (odorant) receptors, second messenger systems, and enzymes (Bowles, E. J., 2003. Chemistry of Aromatherapeutic Oils. Allen & Unwin, ISBN 174114051X; Buchbauer G. Biological activities of essential oils. In: Baser K H C, Buchbauer G, editors. Handbook of Essential Oils: Science, Technology, and Applications. Boca Raton, Fla.: CRC Press; 2010. pp. 235-280). Monoterpenes (C₁₀) and sesquiterpenes (C₁₅) are the classes most commonly identified in Cannabis spp. Terpenoids are the primary aromatic constituents of Cannabis resin, although they constitute only a small percentage of organic solvent extracts (Elsohly et al. Chemical fingerprinting of Cannabis as a means of source identification. Marijuana and cannabinoids pp 51-66. Humana press. 2007).

Without wishing to be bound by theory, it is thought that interplay between the effects of cannabinoids and other compounds derived from Cannabis such as terpenes and/or flavonoids, sometimes referred to as the “entourage effect” can enhance the efficacy of Cannabis extracts for the treatment of a variety of diseases and disorders. For example, it is thought that the terpene myrcene can enhance penetration across the blood brain barrier, pinene can counteract memory and cognition problems, while the combination of pinene, myrcene, and caryophyllene can help treat anxiety.

There are currently at least 80-100 terpenes found in Cannabis. Exemplary terpenes produced by Cannabis that can be extracted using the methods described herein comprise Limonene, Nerolidol, Phytol, Caryophyllene Oxide, Linalool, α-pinene, β-pinene, Eucalyptol, Trans-nerolido, Humulene, delta-3-carene, Camphene, Borneol, Valencene, Geraniol, Myrcene, Terpinolene, β-caryophyllene, selina-3 7(11)-diene, guaiol, 10-epi-y-Eudesmol, β-Eudesmol, α-Eudesmol, Bulnesol, a-Bisabolol, or a combination of any of these. In some embodiments, terpenes extracted using the methods described herein comprise Myrcene, Terpinolene, β-caryophyllene, selina-3 7(11)-diene, guaiol, 10-epi-y-Eudesmol, β-Eudesmol, α-Eudesmol, Bulnesol, α-Bisabolol, or a combination of any of these.

Different Cannabis strains or varieties contain different terpene compositions. For example, strains such as Super Silver Haze, Skywalker and Rock Star produce of beta-caryophyllene. As a further example, strains such as Jack Herer, Strawberry Cough, Blue Dream, Island Sweet Skunk, Dutch Treat and Romulan produce pinenes. As a further example, strains such as Skunk XL, White Widow, and Special Kush produce myrcene. As yet a further example, strains such as Harle-Tsu, Pink Kush, Headband, OG Shark, and ACDC produce α-Bisabolol. The person of ordinary skill will be able to select a Cannabis strain producing the desired terpene(s) for use with the extraction methods disclosed herein.

### Flavonoids

In some embodiments, the instant disclosure provides methods of producing a botanical extract comprising flavonoids. In some embodiments, the botanical extract comprises flavonoids and cannabinoids. In some embodiments, the botanical extract comprises flavonoids, terpenes and cannabinoids.

Flavonoids are secondary polyphenolic metabolites that commonly have a ketone group and yellowish pigments. In Cannabis, at least 20 flavonoids have been identified, mainly belonging to flavone and flavonol subclasses. Without wishing to be bound by theory, it is though that the flavonoids in Cannabis can exert a wide range of biological effects, including aiding in the efficacy of Cannabis extracts for the treatment of diseases or disorders through the entourage effect.

Exemplary flavonoids that can be extracted using the methods of the instant disclosure include, but are not limited to, cannflavin A, cannflavin B, cannflavin C, vitexin, isovitexin, apigenin, kaempferol, quercetin, luteolin, orientin or a combination of any of these.

### Extraction Methods

Cannabis extracts are traditionally made by exposing Cannabis plants to carbon dioxide, butane, propane, alcohol, glycerin, and/or other solvents to leach compounds from Cannabis plants. These techniques often suffer from issues such as high cost, safety issues and/or solvent residues and/or low purity and/or low cannabinoid and terpene extraction efficiency.

Cannabis extraction at cold temperatures with organic solvents has been shown to increase the purity of the product due to the decrease of undesirable components in the final Cannabis extract. However, heat used during the evaporation step to eliminate traces of toxic solvent in the final product alters the cannabinoid and terpene content.

The inventors have found that a highly polyunsaturated lipid-based solvent can be used at cold temperatures (e.g., 0° C. to −40° C.) to extract cannabinoids and/or terpenes from Cannabis plant material. Benefits of these methods include, but are not limited to, one or more of the following (or combinations thereof): it is significantly less expensive and more efficient than supercritical fluid extraction; it is materially safer than extractions using organic solvents; it is faster and safer than traditional methods; and it is capable of producing significantly more potent end products than traditional methods.

The extraction processes of the instant disclosure are useful for the production of Cannabis extracts, whether from marijuana or hemp. The methods of the instant disclosure allow for the production of extracts having more than 75%, more than 80%, more than 90%, more than 95%, more than 96%, more than 97%, more than 98%, more than 98%, more than 99% or more total cannabinoids.

Accordingly, the disclosure provides methods of preparing a botanical extract comprising: (a) providing a plant material in an extraction chamber; (b) contacting a lipid solvent with the plant material; and (c) extracting the plant material with the lipid solvent. In some embodiments, step (c) takes place at −5° C. to −20° C. In some embodiments, step (c) takes place at 0° C. to −40° C.

In some embodiments, the methods of preparing a botanical extract comprise: (a) providing a plant material in an extraction chamber; (b) releasing a lipid solvent from a solvent chamber into the extraction chamber; (c) extracting a bioactive compound from the plant material into the lipid solvent for a first period of time; (d) draining the lipid solvent from the extraction chamber into a cold filtration/centrifugation system; and (e) recovering the lipid solvent from the filtration/centrifugation system thereby producing a botanical extract. In some embodiments, step (c) takes place at −5° C. to −20° C. In some embodiments, step (c) takes place at 0° C. to −40° C.

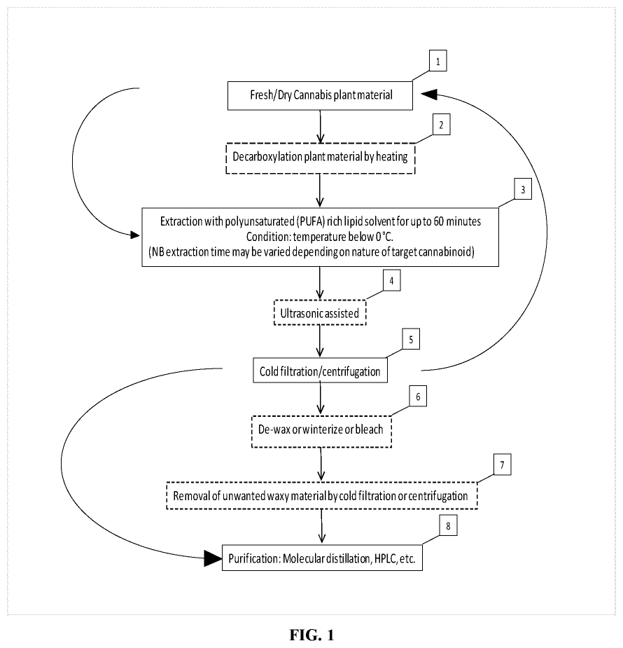

Various steps for producing Cannabis extract are described below. It will be understood that certain steps described can be optional and that the order of steps may vary. FIG. 1 is a flow chart illustrating an example process for producing cannabinoids, terpenes, or flavonoids, using lipid solvent with polyunsaturated fatty acid (PUFA)/saturated fatty acid (SFA) index greater than 7.0 described herein. Exemplary lipid solvents for use in the methods of the invention include, for example, fish oil, flax seed oil, camelina oil, evening primrose oil, black current oil, ahiflower seed oil or a combination thereof

The instant methods use a cold cycle process to combine plant material that is frozen or at room temperature, comprising whole or crushed Cannabis material, with a lipid solvent with a low freezing/melting temperature to produce a botanical extract. In some embodiments, the lipid solvent has a freezing/melting temperature that is below 0° C. In some embodiments, the lipid solvent has a freezing/melting temperature that is between about 0° C. and about −40° C. In some embodiments, the lipid solvent is a PUFA-rich oil with a low freezing/melting temperature (below 0° C.).

Table 1 illustrates exemplary lipid solvents, sometimes referred to herein as oils, that can be used in the methods described herein and their fatty acid profiles.

The method starts, in step 1 of FIG. 1, whereby plant material is placed within an extraction chamber. In some embodiments, the extraction chamber is part of a two chambered extractor comprising an extraction chamber into which the plant material is placed, and a solvent chamber that contains lipid solvent. In some embodiments, the solvent chamber maintains the lipid solvent at a cold temperature, for example between −5° C. and −20° C. The cold lipid solvent is released from the solvent chamber into the extraction chamber where it is left to extract compounds from the plant material. The time of exposure of Cannabis plant material to the lipid solvent (the time period of the extraction step) can be short (up to 60 minutes). In some embodiments, the extraction period is about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes or about 90 minutes. In some embodiments, the extraction period is between about 10 and 60 minutes, between about 10 and 30 minutes, between about 10 and 40 minutes, between about 15 and 40 minutes or between about 15 and 30 minutes. In some embodiments, the extraction step comprises mixing or agitating the plant material and the liquid solvent, for example with a paddle, a stir bar or the equivalent.

In some embodiments, for example those embodiments where it is desired to purify neutral cannabinoids such as THC, CBD, CBN, CBG and CBC, rather than the cannabinoid acids such as THCA, CBDA, CBGA and CBCA, the plant material may be subjected to a decarboxylation step, step 2, prior to step 3, of FIG. 1. The purpose of the decarboxylation step is to convert cannabinoid acids present in the plant material to the neutral cannabinoids. Decarboxylation of cannabinoid acids is a function of time and temperature, thus at higher temperatures a shorter period of time will be taken to complete decarboxylation of a given amount of cannabinoid acid. Suitable conditions may include, for example, a temperature in the range of 135° C. to 145° C. for a time period in the range of 15 to 40 minutes or from 115° C. to 125° C. for a time period in the range of 40 to 75 minutes. Suitable conditions may include 105° C. for 15 minutes, and then 110° C. for a time period in the range of 40 to 75 minutes. Suitable conditions may include 110° C. to 145° C. for a time period in the range of 40 to 75 minutes.

Ultrasound also can be used in an attempt to liberate the cannabinoids from the Cannabis plant, step 4 of FIG. 1. Ultrasound can be produced using sonication. Sonication applies intense shear forces and stress to the plant material and lipid solvent, shearing cell walls and releasing botanical compounds rapidly. An exemplary sonication protocol comprises repeating high pressure and low pressure cycles, for example alternating high pressure and low pressure cycles of 20,000 times per second. Ultrasonication devices will be known to the person of ordinary skill in the art, and are available commercially, for example the Ultrasonicator UP400St from Heischler Ultrasound Technology. As a further example, the 70W (Branson 1510 ultrasonic cleaner) can be used.

After the extraction process is completed, the lipid solvent, which now carries the extracted cannabinoids and terpenes in solution, is drained into a cold filtration/centrifugation system, shown as step 5 of FIG. 1. Suitable solid-liquid filtration centrifuges to filter plant biomass from solvent will be known to the person of ordinary skill in the art. For example, a Model DRC solid-liquid centrifuge available from Rousselet Robatel Kromaton can be used to separate extracted plant material from lipid solvent.

The output lipid solvent may be returned to the reservoir container and recirculated to the extraction chamber to increase the amount of cannabinoids, terpenes, and/or flavonoids. In some embodiments, the output solvent can be returned to the reservoir chamber and fresh plant material extracted at least 2×, 3×, 4×, 5×, 6×, 7×, 8×, 9×, 10× or more times.

In some embodiments, bleaching, dewaxing or winterizing can be done in line, as shown at step 6. Additional unwanted waxy material can be removed by cold filtration or centrifugation, as shown at step 7 of FIG. 1.

Purification of Cannabis oil into cannabinoid distillates may be performed in one or more embodiments under vacuum about 0.001 mbar, by molecular distillation, HPLC, or other methods known to one of ordinary skill in the art, as shown in step 8 of FIG. 1.

Solvents

The disclosure provides lipid solvents for use in the methods of preparing botanical extracts described herein. In some embodiments, the botanical extract comprises a Cannabis extract. Any lipid solvent that is safe for human consumption and that has a suitably low melting point is envisaged as within the scope of the instant disclosure.

In some embodiments, the lipid solvent has a freezing/melting temperature that is below 0° C. In some embodiments, the lipid solvent has a freezing/melting temperature that is between about 0° C. and about −40° C., between about −5° C. and about −40° C., between about −10° C. and about −40° C., between about −15° C. and about −40° C., between about −20° C. and about −40° C., between about −5° C. and about −30° C., between about −10° C. and about −30° C., between about −15° C. and about −30° C., or between about −20° C. and about −30° C. In some embodiments, the lipid solvent has a freezing/melting temperature that is below 0° C., below −5° C., below −10° C., below −20° C., below −25° C., below −30° C., below −35° C., below −40° C. or below −45° C. In some embodiments, the lipid solvent has a freezing/melting point of about −8° C. In some embodiments, the lipid solvent has a freezing/melting point of about −15° C. In some embodiments, the lipid solvent has a freezing/melting point of about −20° C. In some embodiments, the lipid solvent has a freezing/melting point of about −24° C. In some embodiments, the lipid solvent has a freezing/melting point of about −40° C.

In some embodiments, the lipid solvent comprises omega-3 fatty acids. In some embodiments, the lipid solvent comprises monoacylglycerides, diacyglycerides and phospholipids. In some embodiments, the omega-3 fatty acids are omega-3 monoacylglycerides, omega-3 diacyglycerides, omega-3 phospholipids or a combination thereof

As used herein, “glycerides”, also known as “acyglycerols”, refers to a class of molecules where esters are formed between a glycerol and a fatty acid. An “acylglyceride linkage” refers to the covalent bond between the organic acid group, such as a fatty acid, and one of the three hydroxyl groups of the glycerol, for example via an ester linkage.

As used herein, “monoacylglycerides”, or “MAG”, sometimes also referred to as “monoglycerides” or “monoacylglycerols” are a class of glycerides which are composed of a molecule of glycerol linked to a fatty acid via an ester bond. Glycerol contains both primary and secondary alcohol groups. Therefore, two different types of monoglycerides may be formed: 1-monoacylglycerols where the fatty acid is attached to a primary alcohol, and 2-monoacylglycerols where the fatty acid is attached to the secondary alcohol.

“Diacylglycerides”, or “DAG”, sometimes referred to as “diglyceride” or “diacylglycol”, refers to a glyceride consisting of two fatty acids covalently linked to a glycerol molecule through ester linkages. Two possible forms exist: 1,2-diacylglycerols and 1,3-diacylglycerols.

“Triglycerides”, sometimes referred to as “TG”, “TAG”, “triacylglycerol” or “triacylglyceride” are molecules comprising a glycerol linked to three fatty acids via esters.

The term “fatty acid(s)” as used herein refers to long chain aliphatic acids (alkanoic acids) of varying chain lengths, from about C12 to C22 (although both longer and shorter chain-length acids are known). For example, the predominant chain lengths are about C16 to about C22. The structure of a fatty acid is represented by a simple notation system of “X:Y”, where X is the total number of carbon (C) atoms and Y is the number of double bonds.

Generally, fatty acids are classified as saturated or unsaturated. The term “saturated fatty acids” refers to those fatty acids that have no “double bonds” between their carbon backbone. In contrast, “unsaturated fatty acids” are cis-isomers that have “double bonds” along their carbon backbones. “Monounsaturated fatty acids” have only one “double bond” along the carbon backbone (e.g., usually between the 9th and 10th carbon atom as for palmitoleic acid (16:1) and oleic acid (18:1)), while “polyunsaturated fatty acids” (or “PUFAs”) have at least two double bonds along the carbon backbone (e.g., between the 9th and 10th, and 12th and 13th carbon atoms for linoleic acid (18:2); and between the 9th and 10th, 12th and 13th, and 15th and 16th for [alpha]-linolenic acid (18:3)).

PUFAs can be classified into two major families (depending on the position (n) of the first double bond nearest the methyl end of the fatty acid carbon chain). Thus, the “[omega]-6 fatty acids” [omega]-6 or n-6) have the first unsaturated double bond six carbon atoms from the omega (methyl) end of the molecule and additionally have a total of two or more double bonds, with each subsequent unsaturation occurring 3 additional carbon atoms toward the carboxyl end of the molecule. In contrast, the “[omega]-3 fatty acids” ([omega]-3 or n-3) have the first unsaturated double bond three carbon atoms away from the omega end of the molecule and additionally have a total of three or more double bonds, with each subsequent unsaturation occurring 3 additional carbon atoms toward the carboxyl end of the molecule.

A “saturated fatty acid” or “SFA” is a type of fat in which the fatty acid chains have all, or predominantly all, single bonds.

As used herein, “omega-3 fatty acids”, also called “ω-3 fatty acids” or “n-3 fatty acids” refers to polyunsaturated fatty acids (PUFAs) that are characterized by the presence of a double bond three atoms away from the terminal methyl group of the fatty acid. Exemplary omega-3 fatty acids include α-linolenic acid (ALA) found in plant oils, and eicosapentaenoic acid (EPA) and docosahexaenoic acid (DHA), both commonly found in marine oils. Common sources of plant oils containing ALA include walnut, edible seeds, clary sage seed oil, algal oil, flaxseed oil, Sacha Inchi oil, Echium oil, and hemp oil. Common sources of animal omega-3 fatty acids EPA and DHA include fish, fish oils, eggs from chickens fed EPA and DHA, and squid oils.

A “lipid” is a molecule that is soluble in nonpolar solvents. Lipids include fats, faty acids and their derivatives, as well as sterol-containing metabolites such as cholesterols and waxes.

A “phospholipid” refers to a class of lipid comprising two hydrophobic fatty acid tails and a hydrophilic head comprising a phosphate group, which can be joined together via a glycerol molecule. The phosphate groups of the head can be modified with organic molecules such as choline, ethanolamine or serine.

An “omega-3 containing phospholipid” is a phospholipid where one or both of the fatty acid tails of the phospholipid is an omega-3 fatty acid.

In some embodiments, the lipid solvent comprises polyunsaturated fatty acids (PUFA). In some embodiments, the lipid solvent comprises saturated fatty acids (SFA). In some embodiments, the lipid solvent comprises PUFA and SFA. As used herein, the PUFA/SFA index refers to the ratio of PUFA to SFA in the lipid solvent. In some embodiments, the lipid solvent comprises a PUFA/SFA index of at least 7, at least 8, at least 9, at least 10, at least 20, at least 30, at least 40, at least 50, at least 60, at least 70, at least 80, at least 90, at least 100, at least 110, at least 120, at least 130, at least 140 or at least 150.

In some embodiments, the lipid solvent comprises omega-3 fatty acids (Ω-3). Exemplary omega-3 fatty acids include alpha-linolenic acid (ALA), eicosapentaenoic acid (EPA), and docosahexaenoic acid (DHA). ALA is found mainly in plant oils such as flaxseed, soybean, and canola oils. DHA and EPA are found in fish and other seafood. In some embodiments, at least 10%, at least 15%, at least 20%, at least 30%, at least 35%, at least 40%, at least 50%, at least 55%, at 60%, at least 65%, at least 70%, at least 75%, at least 80%, at least 85%, at least 86%, at least 87%, at least 88%, at least 89%, at least 90%, at least 95% or at least 95% of the fatty acids in the lipid solvent are omega-3 fatty acids.

In some embodiments, the lipid solvent comprises fish oil, flax seed oil, camelina oil, evening primrose (EPO) oil, ahiflower seed oil, hemp seed oil, black currant oil, or a combination thereof. In some embodiments, the lipid solvent comprises monoacylglycerides (MAG) and/or diacylglycerides (DAG). In some embodiments, the monoacylglycerides (MAG) and/or diacylglycerides (DAG) are complexed with omega-3 fatty acids. In some embodiments, monoacylglycerides (MAG) and/or diacylglycerides (DAG) are complexed with polyunsaturated omega-3 fatty acids. In fish oils, MAG and DAG are naturally present in trace amounts. However, in concentrated fish oils that have converted ethyl ester fatty acids to TAG fatty acids (known as re-esterified triglycerides), the amount of MAG and DAG in the product can be higher due to incomplete enzymatic or chemical reactions. In some embodiments, for example those embodiments where the lipid solvent comprises an animal oil, the lipid solvent comprises about 1% to about 3% MAG. In some embodiments, for example those embodiments where the lipid solvent is a plant oil, the lipid solvent comprises about 0.2% to about 3% MAG. In some embodiments, the lipid solvent comprises less than 3%, less than 2.5%, less than 2%, less than 1.5%, less than 1%, less than 0.5%, less than 0.4%, less than 0.3%, less than 0.2%, or less than 0.1% MAG.

In some embodiments, the lipid solvent comprises about 0.5% to about 40% DAG. In some embodiments, for example those embodiments where the lipid solvent comprises an animal oil, the lipid solvent comprises about 1% to about 40% DAG. In some embodiments, for example those embodiments where the lipid solvent comprises a plant oil, the lipid solvent comprises between 0.5% to about 7% DAG. In some embodiments, the lipid solvent comprises less than 40%, less than 30%, less than 20%, less than 10%, less than 9%, less than 8%, less than 7%, less than 6%, less than 5%, less than 4%, less than 3%, less than 2%, less than 1%, less than 0.7%, less than 0.5%, less than 0.3%, or less than 0.2% DAG. In some embodiments, the lipid solvent comprises a fish oil, and the fish oil comprises between about 1% and about 3% MAG, and between about 1% and about 40% DAG. In some embodiments, the lipid solvent comprises a vegetable oil, and the vegetable oil comprises between about 0.2% and about 3.0% MAG, and between about 0.5% and about 7.0% DAG.

TABLE 1
Fatty acid profiles of exemplary oils
with a freezing point below −5° C.

| | | | | PUFA/ | |
|---|---|---|---|---|---|
| | | ω-3 (as | % MAG; | SFA | Freezing |
| Oil Type | Solvent Name | % FA) | % DAG² | Index | Point |
| Animal | EE Fish Oil | 88 | 1-3% MAG; | 46.3 | −40° C. |
| Oil | RTG Fish Oil | 75 | 1-40% DAG | ≥100 | |
| Flax seed | Omega Nutrition | 55 | 0.2-3% | 8.9 | |
| oil (from | flax seed oil | | MAG; | | |
| different | TAFOODs flax | 57 | 0.5-7% | 10.7 | |
| sources) | seed oil | | DAG | | |
| | Shape Foods | 66 | | 10.1 | −24° C. |
| | High ALA Flax | | | | |
| | oil | | | | |
| | Shape Foods | 57 | | 9.1 | |
| | Organic Cold | | | | |
| | press | | | | |
| Camelina¹ | Camelina oil | 35 | | 7.3 | −15° C. |
| oil | | | | | |
| EPO oil | EPO | ≥9 | | 10.3 | −20° C. |
| Ahiflower | Natures Crops | 66 | | 11.5 | −20° C. |
| seed oil | Ahiflower oil | | | | |
| Hemp | Hemp seed oil | 18 | | 8.2 | −8° C. |
| seed oil | Chii | | | | |
| Black | Black currant oil | 15 | | 9.1 | −20° C. |
| currant oil | | | | | |

Abbreviations:
ω, omega;
ALA, alpha-linolenic acid;
DAG, diacylglyceride;
EPO, evening primrose oil;
FA, fatty acid;
MAG, monoacylglyceride;
PUFA, polyunsaturated fatty acid;
RTG, re-esterified triglyceride;
SFA, saturated fatty acid
¹Data from Health Canada
²indicates percent glycerides that are MAG and that are DAG

### Decarboxylation

In some embodiments, Cannabis plant material used in the extraction methods described herein is decarboxylated. Decarboxylation is a chemical reaction that converts an acid to a phenol, and releases carbon dioxide (CO₂), thereby removing a carbon atom from a carbon chain. Most cannabinoids exist as acids and neutral (i.e. decarboxylated) forms. Phytocannabinoids are synthesized in the plant as acid forms. Some decarboxylation does occur in the Cannabis plant. However, decarboxylation increases significantly after the plant is harvested, and the kinetics of decarboxylation increase at higher temperatures than found in vivo.

All methods of decarboxylation known in the art are envisaged as within the scope of the instant disclosure. Exemplary decarboxylation methods are described in U.S. Pat. No. 7,344,736, the contents of which are incorporated herein by reference in their entirety.

The decarboxylation step may be carried out prior to or after extraction with lipid solvent.

In some embodiments, the decarboxylation step is carried out prior to extraction with lipid solvent and is conducted by heating the Cannabis plant material to temperatures and for times which ensure at least 95% conversion of the acid cannabinoids from the acid form to their neutral form, while ensuring thermal degradation of THC to CBN is less than 10%.

Decarboxylation of cannabinoid acids is a function of time and temperature, thus at higher temperatures a shorter period of time will be taken for complete decarboxylation of a given amount of cannabinoid acid. In selecting appropriate conditions for decarboxylation consideration must, however, be given to minimizing thermal degradation of the desirable, pharmacological cannabinoids into undesirable degradation products, for example thermal degradation of THC to cannabinol (CBN).

In some embodiments, decarboxylation is carried out in a multi-step heating process in which the plant material is first heated to a first temperature for a first (relatively short) time period to evaporate off retained water and allow for uniform heating of the plant material; and second the temperature is increased to a second temperature for a second time period (typically longer than the first time period) until at least 95% conversion of the acid cannabinoids to their neutral form has occurred.

In some embodiments, the first step is conducted at a temperature in the range of 100° C. to 110° C. for 10-20 minutes. In some embodiments, the first temperature is about 105° C. and the first time period is about 15 minutes.

If the plant material is derived from Cannabis plants having a high CBD content, the second temperature can be in the range from 115° C. to 125° C., for example about 120° C. and the second time period is in the range from 45 to 75 minutes, for example about 60 minutes. In some embodiments, the second temperature is in the range from 135° C. to 145° C., for example 140° C. and the second time period is in the range from 15 to 45 minutes, for example about about 30 minutes.

If the plant material is derived from Cannabis plants having a high THC content, the second temperature is can be in the range of 115° C. to 125° C., for example 120° C., and the second time period can be in the range of 45 minutes to 75 minutes, for example about 60 minutes. In some embodiments, the second temperature is in the range of 100° C. to 110° C., for example 105° C., and the second time period is in the range of 60 to 120 minutes.

In some embodiments, the decarboxylation step is conducted at temperatures and for times which ensure at least 97% conversion of the acid cannabinoids to their neutral form, while ensuring thermal degradation of THC to CBN is less than 5%.

In some embodiments, decarboxylation is carried out in 2 steps, for example 105° C. for 15 minutes, and then at 110° C. for about 40 to about 70 minutes.

In some embodiments, decarboxylation is carried out in a single step heating process in which the plant material is heated to between about 115° C. to 145° C. In some embodiments, decarboxylation is carried out in a single step heating process in which the plant material is heated to between about 110° C. to 145° C. In some embodiments, decarboxylation is carried out at about 110° C. or 115° C. In some embodiments the plant material is heated to between about 110° C. to 145° C. for less than 15 minutes, less than 30 minutes, less than 45 minutes, less than 60 minutes, less than 75 minutes, less than 90 minutes, less than 105 minutes or less than 120 minutes. In some embodiments the plant material is heated to between about 110° C. to 145° C. for less than one hour. In some embodiments the plant material is heated to between about 110° C. to 145° C. for between about 30 and 60 minutes.

In some embodiments, the decarboxylation step is carried out after extraction with lipid solvent.

### Bleaching

In some embodiments, the methods described herein comprise bleaching the botanical extract. As used herein, “bleaching” refers to a process of removing undesired minor impurities from a botanical extract, such as color pigments, free fatty acids, peroxides, undesired odor causing compounds and non-fatty materials.

In some embodiments, bleaching comprises contacting the botanical extract with a bleaching agent. Exemplary bleaching agents include natural earth clay, bentonite, acid activated clay, silica gel, diatomaceous earth, bleaching earth, activated carbon, mixtures of magnesium oxide and alumina zeolitic, or combinations thereof. For example, the botanical extract can be filtered through a cake of bleaching agent and a filter using a vacuum.

### Winterizing and De-waxing

In some embodiments, the methods of preparing a Cannabis extract described herein comprise winterization and/or de-waxing. Winterization and de-waxing are methods to remove undesired Cannabis lipids and waxes from Cannabis extracts. Winterization can be achieved by dissolving a non-polar substance (e.g., the cannabinoid extract) into a polar solvent (e.g. ethanol) at sub-zero temperatures. This separates waxes and lipids from the cannabinoid extract, forcing them to collect at the top of the mixture for easy filtration.

An exemplary winterization method is described in U.S. Pat. No. 7,344,736. Ethanol is added to the Cannabis extract in the ratio of 2:1 ethanol volume to weight. The ethanolic solution is then cooled to −20° C.±5 ° C. and held at this temperature for approximately 48 hours. On completion of the winterization, the precipitated waxes and lipids are removed by cold filtration through a 20 μm filter.

De-waxing also uses low temperatures to separate waxes and lipids from Cannabis extract. In de-waxing, Cannabis extract mixed with a solvent such as butane is cooled to low temperatures (e.g. −20° C. or below) which makes the waxes and lipids insoluble in the butane solution. Once the waxes and undesired lipids have separated from the solvent, the mixture is passed through a variety of micron screens, effectively filtering out all undesired waxes and lipids. An exemplary de-waxing protocol comprises chilling the Cannabis extract and butane composition to low temperatures, then running the composition through a Buchner funnel that is attached to a passive vacuum, thus filtering out waxes and lips and producing a pure final product. The filtered product is then passed to a heated chamber where the butane can be removed through evaporation.

### Purification of Botanical Extracts

Additional purification methods that can be applied to Cannabis extracts produced using the methods described herein will be known to the person of ordinary skill in the art.

Exemplary additional purification methods are described in EP 1385595 B1 and U.S. Pat. No. 7,344,736, the contents of which are incorporated by reference in their entirety.

In some embodiments, partially purified botanical extracts may be further purified by chromatographic separation. High performance liquid chromatography (HPLC) is an analytical technique for determination and assay of constituents and can be used in preparative mode to produce quantities of concentrated fractions and individual components. HPLC uses pumps to pass a pressurized liquid solvent containing the botanical extract through a column filled with a solid adsorbent material. Each component of the botanical extract, such as different terpenes, flavonoids or cannabinoids, interacts slightly differently with the adsorbent material, causing different flow rates for the different components and leading to the separation of the components as they flow out of the column. However, HPLC is subject to limitations of scale as a production technique and there remains a need for additional methods of separation to produce large-scale quantities of plant extracts of sufficient quality for formulation into pharmaceutical dosage forms.

In some embodiments, distillation and/or sublimation can be used to further purify Cannabis extracts of the instant disclosure. Distillation and sublimation have been used to separate components of plant medicines which have boiling points at or around the temperature at which water boils at atmospheric pressure (100° C.). Separation by distillation is a physical process widely used in the preparation of essential oils. For example, GB 635,121 describes a process for the preparation of extracts from aromatic plants by distillation with the help of a hot gas, preferably under high vacuum. As a further example, WO 99/11311 describes a vaporizer for inhalation and a method for the extraction of active ingredients from a crude natural product. This method utilizes an ascending stream of hot air, or a heated inert gas stream, to volatilize components from the natural product. The resultant vapor may then be inhaled by a user. As yet a further example, WO00/25127 is concerned with a method of preparing tetrahydrocannabinol using extraction of plant material with a non-polar solvent followed by vacuum distillation and collection of a constant boiling fraction. Additional distillation steps and chromatographic steps, including HPLC, reverse phase HPLC and flash chromatography, may be performed.

In some embodiments, molecular distillation can be used to further purify Cannabis extracts of the instant disclosure. Molecular distillation, sometimes called short path distillation, is a separation technique that separates compounds through a process of slow thermal heating. The compounds in Cannabis extracts, such as cannabinoids, terpenes and flavonoids, have different vapor pressure points (boiling points). Through precise temperature control of the distillation process, molecular distillation can separate a Cannabis extract into one or more high-purity fractions. In exemplary embodiments, the final materials produced through short path distillation include one or more cannabinoids, one or more terpenes, and optionally, any leftover waxes, sugars, and heavy residues. In some embodiments, the molecular distillation comprises more than one round of molecular distillation.

In some embodiments, Cannabis extracts produced by the methods of the instant disclosure can be further purified using column chromatography. Column chromatography is a method use to separate compounds based on differential absorption of the compounds to the adsorbent packed in a column. The compounds, such as different terpenes, flavonoids and cannabinoids move through the column at different rates, allowing them to be separated into fractions. The column chromatography can be carried out using any known packing material including, for example, silica or alumina for normal phase operation or Ciβ or Cβ bonded phase silica for reversed phase operation. Elution of the normal phase chromatography column is carried out with solvents having an increasing polarity. Non-polar solvents include the lower straight chain and branched chain alkanes, including, for example, pentane, hexane, isooctane and petroleum ether. More polar solvents include various organic ethers, alcohols, esters or ketones, including, for example dialkyl ethers, lower alkyl acetates, lower dialkyl ketones and lower alkanols. Illustrative polar solvents include, for example, acetone, ethylacetate, diethylether and isopropyl alcohol. The ratio of non-polar solvent to polar solvent can vary between 100:0 to 80:20.

### Botanical Extracts and Compositions

The disclosure provides botanical extracts produced using the methods described herein. The botanical extracts can comprise at least one bioactive molecule derived from Cannabis, such as cannabinoids, terpenes or flavonoids, and a solvent. Alternatively, or in addition, the botanical extracts produced using the methods described herein may be formulated as resins.

The disclosure provides compositions comprising the botanical extracts produced using the methods described herein and a pharmaceutically acceptable carrier, diluent or excipient.

As used herein, a “botanical extract” refers to a composition comprising components extracted from plant material.

In some embodiments, the botanical extract comprises a resin. In some embodiments, the resin comprises one or more cannabinoids. In some embodiments, the resin comprises one or more cannabinoids and one or more terpenes. In some embodiments, the resin comprises one or more cannabinoids, one or more terpenes and/or one or more flavonoids.

In some embodiments, the botanical extract comprises a solid, for example a precipitate or crystalized form or the extract. In some embodiments, the botanical extract is a powder. Powders of the botanical extracts of the disclosure can be generated by methods such as spray drying, or by the addition of a plating agent or other additive that can act as a carrier. Spray drying is a method of producing a powder from a liquid or slurry by rapidly drying with hot gas. Exemplary plating agents include N-ZORBIT 2144 DG. In some embodiments, the botanical extract is formulated as a powder and comprises a plating agent or carrier. Powders of desired particle size can be generated by milling, which subjects particles to mechanical stress, breaking the particles into smaller sizes.

In some embodiments, the botanical extract comprises a liquid, for example a liquid comprising one or more cannabinoids or other bioactive molecules extracted from Cannabis and a lipid solvent. In some embodiments, the botanical extract comprises one or more cannabinoids, one or more terpenes and a lipid solvent. In some embodiments, the botanical extract comprises one or more cannabinoids, one or more flavonoids and a lipid solvent. In some embodiments, the botanical extract comprises one or more cannabinoids, one or more terpenes, one or more flavonoids and a lipid solvent. In some embodiments, the lipid solvent comprises fish oil, flax seed oil, camelina oil, evening primrose (EPO) oil, ahiflower seed oil, hemp seed oil, black currant oil, or a combination thereof. In some embodiments, the lipid solvent comprises less than or equal to 3% MAG.

In some embodiments, the botanical extract comprises at least 75% cannabinoids, at least 80% cannabinoids, at least 85% cannabinoids, at least 90% cannabinoids, at least 95% cannabinoids, at least 96% cannabinoids, at least 97% cannabinoids, at least 98% cannabinoids or at least 99% cannabinoids.

The disclosure provides compositions comprising the botanical extracts produced using the methods described herein. The disclosure provides compositions comprising (a) a Cannabis extract produced using the methods described herein, wherein the extract comprises at least one cannabinoid, and (b) a pharmaceutically acceptable carrier, diluent or excipient.

In some embodiments, the at least one cannabinoid comprises Δ9-tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof.

In some embodiments, the at least one cannabinoid comprises a combination of THC and CBD.

In some embodiments, the at least one cannabinoid comprises a combination of THC, THCA, CBD and CBDA.

In some embodiments, the at least one terpene comprises myrcene, terpinolene, β-caryophyllene, selina-3 7(11)-diene, guaiol, 10-epi-y-eudesmol, β-eudesmol, a-eudesmol, bulnesol, α-bisabolol or a combination thereof

In some embodiments, the at least one flavonoid comprises cannflavin A, cannflavin B, cannflavin C, vitexin, isovitexin, apigenin, kaempferol, quercetin, luteolin, orientin or a combination thereof.

In some embodiments, the composition comprises about 2% to about 50% cannabinoids, about 2% to about 20% cannabinoids, about 2% to about 40% cannabinoids, about 2% to about 30% cannabinoids, about 2% to about 20% cannabinoids, about 2% to about 15% cannabinoids, 5% to about 50% cannabinoids, about 5% to about 20% cannabinoids, about 5% to about 40% cannabinoids, about 5% to about 30% cannabinoids, about 5% to about 20% cannabinoids, about 5% to about 15% cannabinoids, 10% to about 50% cannabinoids, about 10% to about 20% cannabinoids, about 10% to about 40% cannabinoids, about 10% to about 30% cannabinoids, about 10% to about 20% cannabinoids or about 10% to about 15% cannabinoids.

In some embodiments, the composition comprises about 2% to 20% cannabinoids. In some embodiments, the composition comprises about 5% to 20% cannabinoids. In some embodiments, the composition comprises about 5% to 15% cannabinoids.

In some embodiments, the composition comprises at least one cannabinoid and a pharmaceutically acceptable carrier, diluent or excipient. In some embodiments, the composition comprises at least one cannabinoid, at least one terpene, and a pharmaceutically acceptable carrier, diluent or excipient. In some embodiments, the composition comprises at least one cannabinoid, at least one terpene, at least one flavonoid and a pharmaceutically acceptable carrier, diluent or excipient. In some embodiments, the at least one terpene comprises myrcene, terpinolene, β-caryophyllene, selina-3 7(11)-diene, guaiol, 10-epi-y-eudesmol, β-eudesmol, α-eudesmol, bulnesol, α-bisabolol or a combination thereof. In some embodiments, the at least one flavonoid comprises cannflavin A, cannflavin B, cannflavin C, vitexin, isovitexin, apigenin, kaempferol, quercetin, luteolin, orientin or a combination thereof.

In some embodiments, the composition comprises an antioxidant such as alpha-tocopherol, a mixture of tocopherols, or rosemary extract.

Any pharmaceutically acceptable carrier, diluent or excipient known in the art can be used in the Cannabis extract compositions described herein. Examples of pharmaceutically acceptable carriers, diluents and excipients for oral delivery include: sodium bicarbonate solutions and similar diluents which neutralize stomach acid or have similar buffering capacity, glycols, oils or emulsions; and include formulations in the form of gels, pastes and viscous colloidal dispersions. The Cannabis extract compositions may be presented in capsule, tablet, slow release or elixir form or as a gel or paste. Furthermore, the Cannabis extract compositions may be presented as a food or drink.

Suitable carriers or diluents illustratively include, but are not limited to, either individually or in combination, lactose, including anhydrous lactose and lactose monohydrate; starches, including directly compressible starch and hydrolyzed starches; mannitol; sorbitol; xylitol; dextrose and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of alpha- and amorphous cellulose, powdered cellulose, and hydroxypropylmethylcellulose (HPMC); calcium carbonate; glycine; bentonite; block co-polymers; polyvinylpyrrolidone; and the like.

Cannabis extract compositions of the disclosure optionally comprise one or more pharmaceutically acceptable disintegrants as excipients, particularly for tablet formulations. Suitable disintegrants include, but are not limited to, either individually or in combination, starches, including sodium starch glycolate and pregelatinized corn starches, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums.

Cannabis extract compositions of the disclosure optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, but are not limited to, either individually or in combination, acacia; tragacanth; sucrose; gelatin; glucose; starches such as, but not limited to, pregelatinized starches; celluloses such as, but not limited to, methylcellulose and carmellose sodium Tylose; alginic acid and salts of alginic acid; magnesium aluminum silicate; polyethylene glycol (PEG); guar gum; polysaccharide acids; bentonites; povidone, for example povidone K-15, K-30 and K-29/32; polymethacrylates; hydroxypropylcellulose; and ethylcellulose.

Polymeric binding agents can have varying molecular weight, degrees of crosslinking, and grades of polymer. Polymeric binding agents can also be copolymers, such as block copolymers that contain mixtures of ethylene oxide and propylene oxide units. Variation in these units' ratios in a given polymer affects properties and performance. Examples of block co-polymers with varying compositions of block units are Poloxamer 188 and Poloxamer 237 (BASF Corporation).

Cannabis extract compositions of the disclosure optionally comprise one or more pharmaceutically acceptable wetting agents as excipients. Non-limiting examples of surfactants that can be used as wetting agents in Cannabis extract compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol and octoxynol 9, poloxamers (polyoxyethylene and polyoxypropylene block copolymers, polyoxyethylene fatty acid glycerides and oils, for example polyoxyethylene caprylic/capric mono- and diglycerides, polyoxyethylene, castor oil and polyoxyethylene, hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate and polysorbate, Tween 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof.

Cannabis extract compositions of the disclosure optionally comprise one or more pharmaceutically acceptable lubricants (including anti-adherents and/or glidants) as excipients. Suitable lubricants include, but are not limited to, either individually or in combination, glyceryl behapate (Compritol 888); stearic acid and salts thereof, including magnesium, calcium and sodium stearates; hydrogenated vegetable oils; colloidal silica; talc; waxes; boric acid; sodium benzoate; sodium acetate; sodium fumarate; sodium chloride; DL-leucine; PEG Carbowax; sodium oleate; sodium lauryl sulfate; and magnesium lauryl sulfate.

Suitable anti-adherents include, but are not limited to, talc, cornstarch, DL-leucine, sodium lauryl sulfate and metallic stearates.

Glidants can be used to promote powder flow of a solid formulation. Suitable glidants include, but are not limited to, colloidal silicon dioxide, starch, talc, tribasic calcium phosphate, powdered cellulose and magnesium trisilicate. Colloidal silicon dioxide is particularly preferred. Other excipients such as colorants, flavors and sweeteners are known in the pharmaceutical art and can be used in Cannabis extract compositions of the instant disclosure. Tablets can be coated, for example with an enteric coating, or uncoated. Compositions of the invention can further comprise, for example, buffering agents.

Cannabis extract compositions of the instant disclosure may also contain additives, such as water, alcohols, oils (mineral, vegetable, animal and synthetics), glycols, colorants, preservatives, emulsifiers, gelling agents, gums, esters, hormones, steroids, anti-oxidants, silicones, polymers, fragrances, flavors, other active ingredients, acids, bases, buffers, vitamins, minerals, salts, polyols, proteins and their derivatives, essential oils, other enzymes, co-enzymes and extracts, surfactants, detergents, soaps, anionics, non-ionics, ionics, waxes, lipids, stabilizers, fillers, celluloses, glycans, amines, solubilizers, thickeners, sugars and sugar derivatives, ceramides, sweeteners and the like, so long as such additives do not defeat the objectives of the present invention.

Cannabis extract compositions of the disclosure may be formulated for topical administration. For example, Cannabis extract compositions may be formulated as a liquid, gel, cream, ointment, lotion, salve, balm or paste. Topical formulations can comprise pharmaceutically acceptable carriers, solvents, adhesives, dispersion agents and the like. Topical formulations can be formulated for application to intact skin or mucous membranes, and have a highly localized effect.

Cannabis extract compositions of the disclosure may be formulated for transmucosal administration, parenteral administration, subdermal administration, or inhalation. For example, Cannabis extract compositions can be injected intravenously or under the skin (subcutaneously, or subdermal administration).

Cannabis extract compositions of the disclosure may be formulated for transmucosal administration. For example, transmucosal administration can encompass oral formulations for buccal administration, and aerosol sprays for nasal administration and/or inhalation.

Cannabis extract compositions of the disclosure may be formulated for inhalation. For example, Cannabis extract compositions can be formulated as vapors or aerosols that can be inhaled into the lungs. Vapor formulations include liquid formulations that are vaporized when loaded into a suitable vaporization device.

### Antioxidants

The disclosure provides compositions comprising a Cannabis extract, a pharmaceutically acceptable carrier and an antioxidant.

In some embodiments, the anti-oxidant is a fat-soluble antioxidant. Antioxidants are compounds that inhibit oxidation, a chemical reaction that can produce free radicals, which can cause cellular damage.

In some embodiments, the antioxidant comprises alpha tocopherol, a mixture of tocopherols, or rosemary extract. Exemplary tocopherols include d-α-tocopheryl acetate, d-α-tocopheryl acid succinate, d-β-tocopherol, d-β-tocopherol, d-γ-tocopherol, d-δ-tocopherol, d-α-tocotrienol, d-β-tocotrienol, d-γ-tocotrienol, d-δ-tocotrienol, dl-a-tocopherol, dl-α-tocopheryl acetate, dl-α-tocopheryl calcium succinate, dl-α-tocopheryl nicotinate, dl-α-tocopheryl linoleate/oleate and all other possible stereo isomeric forms of the above compounds, and are sometimes referred to as “Vitamin E.” Additional anti-oxidants include beta-carotene, carotenoids, and Vitamin A.

In some embodiments, the anti-oxidant comprises astaxanthin.

### Formulation for Oral Administration

In some embodiments, the composition is formulated for oral administration. An oral composition according to the instant disclosure may be in any of the dosage forms which are generally used for dietary supplements such as liquids, gels, powders, tablets, caplets, capsules, gelcaps, food additives, drops, beverages, pills, lozenges, rinses, pastes, gums and soft gels.

### Methods of Making Cannabis Extract Compositions

The disclosure provides methods of making the compositions comprising the botanical extract described herein. In some embodiments, the methods comprise (a) providing a Cannabis extract produced using the methods described herein; and (b) mixing the Cannabis extract with a pharmaceutically acceptable carrier, diluent or excipient. In some embodiments, the methods comprise mixing the Cannabis extract and the pharmaceutically acceptable carrier with one or more antioxidants.

In some embodiments, Cannabis extract comprises a liquid or a resin.

In some embodiments, the Cannabis extract is formulated with a pharmaceutically acceptable carrier, diluent or excipient. The pharmaceutically acceptable carrier, diluent or excipient can be a liquid, for example a liquid comprising fish oil, flax seed oil, camelina oil, evening primrose oil, black current oil, ahiflower seed oil, or a combination thereof.

In some embodiments, the Cannabis extract is mixed with the pharmaceutically acceptable carrier, diluent or excipient at a ratio of about 1:7, about 1:8, about 1:9, about 1:9.5, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, or about 1:25 Cannabis extract to pharmaceutically acceptable carrier. In some embodiments, the Cannabis extract is mixed with the pharmaceutically acceptable carrier at a ratio of about 1:9 Cannabis extract to pharmaceutically acceptable carrier.

### Other Embodiments

While the invention has been described in conjunction with the detailed description thereof, the foregoing description is intended to illustrate and not to limit the scope of the invention, which is defined by the scope of the appended claims. Other aspects, advantages, and modifications are within the scope of the following.

### EXAMPLES

### Example 1

### Extraction of Dry Cannabis with Cold Flax seed Oil

Step 1: 10 grams (g) of dry, frozen flowers of whole Cannabis from strain Nebula, with 10% CBDA and 5.6% THCA by weight percent, were placed in an open vessel.

Step 2: 200 g of cold flax seed oil (−5° C. to −20° C.) was added to the same vessel.

Step 3: The contents of said vessel were stirred at cold temperature (−5° C. to −20° C.) for about 10 to 30 minutes.

Optional Step 4: An ultrasound device can be used in vessel of extraction. With power ultrasound, Cannabis extraction is faster and highly efficient.

Step 5: The contents of the vessel were then cold-filtered or cold-centrifuged at 100×g for 5 minutes to remove solid materials.

Optional Step: Step 2 to 5 can be repeated with the same oil until the concentration of cannabinoids in the oil reaches the target concentration. Table 2 illustrates the extraction efficiencies of active Nebula ingredients with cold flax seed oil.

Optional Step: Additional purification processes can be applied to the extract.

TABLE 2
Relative extraction efficiency of dry cold crushed Nebula
flowers extracted with cold flax seed oil.

| | THC + | CBD + | Total | |
|---|---|---|---|---|
| | THCA | CBDA | Cannabinoid | Terpenes |
| −10° C., 10 min | 40.4 | 51.1 | 47.3 | 144.2 |
| −10° C., 30 min | 71.8 | 85.4 | 80.7 | 185.7 |

Table 2 shows the relative extraction efficiency (%) of active ingredients of dry cold crushed Nebula flowers extracted with cold flax seed oil (−10° C.) during 10 minutes and 30 minutes. The control extraction was done with Methanol/Chloroform (9:1). Extraction efficiency (%) is relative to the control extraction.

### Example 2

### Extraction of Dry Cannabis with Cold Fish Oil

Step 1: 10 g of dry, frozen crushed Cannabis flowers from strain Nebula, with 10% CBDA and 5.6% THCA by weight percent, were placed in an open vessel.

Step 2: 100 g of cold fish oil (−5° C. to −20° C.) was added to the same vessel.

Step 3: The contents of said vessel were stirred at cold temperature (−5° C. to −20° C.) for about 10 to 30 minutes.

Optional Step 4: An ultrasound device can be used in vessel of extraction. With power ultrasound, Cannabis extraction is faster and highly efficient.

Step 5: The contents of the vessel were then cold-filtered or cold-centrifuged at 100×g for 5 minutes to remove solid materials.

Optional Step: Steps 2 to 5 can be repeated using the same oil until the concentration of cannabinoids in the oil reaches the target concentration. Table 3 illustrates the extraction yield of 1 simple extraction versus 3 extractions using recycled fish oil.

Optional Step: Additional purification processes can be applied to the extract.

TABLE 3
Comparison of the extraction yield of one extraction or
extracting three times using recycled Fish Oil

| | | Single | Triple Extract with |
|---|---|---|---|
| Parameter | Units | extract | recycled solvent |
| Dry cannabis extracted | g | 10 | 30 (3 × 10) |
| by 100 g solvent | | | |
| Total cannabinoids | mg/g | 15.7 | 42.8 |
| (neutral form) | final oil | | |
| Total THC | | 4.7 | 12.9 |
| Total CBD | | 10.1 | 26.0 |
| Total CBG | | 0.6 | 3.7 |
| Terpenes | | 0.2 | 3.6 |

### Example 3

### Extraction of Fresh Cannabis with Cold Flax Seed Oil

Step 1: 10 g of fresh, frozen, crushed Cannabis flowers from strain Nebula, with 10% CBDA and 5.6% THCA by weight percent, were placed in an open vessel.

Step 2: 100 g of cold flax seed oil (−5° C. to −20° C.) was added to the same vessel.

Step 3: The contents of said vessel were stirred at cold temperature (−5° C. to −20° C.) for about 10 to 30 minutes.

Optional Step 4: An ultrasound device can be used in vessel of extraction. With power ultrasound, Cannabis extraction is faster and highly efficient.

Step 5: The contents were then cold-filtered or cold-centrifuged at 100×g for 5 minutes to remove solid materials.

Optional Step: Repeat step 2 to 5 until the concentration of cannabinoids in the oil reaches the target.

Optional Step: Additional purification processes can be applied to the extract.

Fresh Cannabis flowers contain only acidic forms of cannabinoids. Extraction with organic solvent cause cannabinoid alteration. Without wishing to be bound by theory, this is probably due to the evaporation step. With lipid extraction the inventors obtain a representative extract of the Cannabis in its natural state. Table 4 illustrates the integrity of cannabinoids in fresh, cold, and crushed material extracted with different exemplary solvents using methods of the instant disclosure. Table 5 shows % terpenes extraction efficiency with various exemplary solvents from cold, crushed, fresh flowers.

TABLE 4
Integrity of Cannabinoids from fresh, cold, crushed material extracted with different solvents

| Solvent used | % THCA | % THC | % CBDA | % CBD | % CBGA | % CBG | % CBC |
|---|---|---|---|---|---|---|---|
| Fresh flowers NC/Nebula strains* | 31.8 | 0 | 64.1 | 0 | 4.1 | 0 | 0 |
| Cold Ethanol extract, 30 min. | 27.9 | 5.1 | 57.9 | 5 | 3.8 | 0 | 0.3 |
| Cold Acetone extract, 30 min. | 32.5 | 1 | 55.4 | 1.4 | 4.7 | 0 | 0 |
| Flax seed oil extract, 10 min. | 31.1 | 0 | 64.7 | 0 | 4.1 | 0 | 0 |
| Flax seed oil extract, 30 min. | 31.3 | 0 | 65.1 | 0 | 3.6 | 0 | 0 |

*Control extraction, which was carried out Methanol/Chloroform 9:1

TABLE 5
Relative percentage of Terpenes extraction efficiency on fresh
cold crushed material with different solvents (compared
with control extraction using Methanol/Chloroform at 9:1)

| | Cold ethanol | Cold acetone | Flax seed oil |
|---|---|---|---|
| Parameters | extract, 30 min | extract, 30 min | extract, 30 min |
| Myrcene | 0 | 0.1 | 226.7 |
| Terpinolene | 0.2 | 0.6 | 219.4 |
| β-caryophyllene | 11.4 | 13.7 | 208.2 |
| selina-3 7(11)-diene | 11.8 | 14.1 | 140.7 |
| Guaiol | 14.2 | 16 | 168.9 |
| 10-epi-y-Eudesmol | 15.2 | 16.9 | 166.5 |
| β-Eudesmol | 18.6 | 22.5 | 140.7 |
| α-Eudesmol | 6.8 | 7 | 136.8 |
| Bulnesol | 11.2 | 11 | 164.2 |
| cc-Bisabolol | 13.2 | 14.4 | 166.5 |
| Total Terpenes | 3.1 | 3.6 | 220.5 |

### EXAMPLE 4

### Extraction of Dry, Frozen, Crushed Cannabis Flowers with Cold Ahiflower Seed Oil

Step 1: 20 g of dry, frozen, crushed Cannabis flowers from strain Nebula, with 9.8% CBD+CBDA and 4.6% THC+THCA by weight percent, were placed in an open vessel.

Step 2: 200 g of cold ahiflower seed oil (−10° C.) was added to the same vessel.

Step 3: The contents of said vessel were stirred at cold temperature (−5° C. to −10° C.) for about 30 to 60 minutes.

Optional Step 4: An ultrasound device can be used in vessel of extraction. With power ultrasound, Cannabis extraction is faster and highly efficient.

Step 5: The contents were then cold-filtered or cold-centrifuged at 100×g for 5 minutes to remove solid materials. Table 6 illustrates the extraction efficiencies of Nebula active ingredients with cold ahiflower seed oil.

Optional Step: Additional purification processes can be applied on the extract.

TABLE 6
Relative extraction efficiency (%) of active ingredients
with cold ahiflower seed oil.

| | THC + | CBD + | Total | |
|---|---|---|---|---|
| | THCA | CBDA | cannabinoids | Terpenes |
| −5° C., 30 minutes | 91.9 | 101.7 | 101.8 | 286.1 |
| −5° C., 60 minutes | 91.9 | 101.7 | 101.8 | 261.6 |

Table 6 shows the relative extraction efficiency (%) of active ingredients of dry cold crushed Nebula flowers with cold ahiflower seed oil at −5° C. during 30 minutes and 60 minutes. The control extraction was done with Methanol/Chloroform (9:1). Extraction efficiency (%) is relative to the control extraction.

### EXAMPLE 5

### Chlorophyll Content of Lipid Extracts

Dry Cannabis flowers contain large amounts of chlorophyll, which can be undesirable in Cannabis extracts.

Intact or ground Cannabis was extracted with ahiflower seed or camelina seed oil using the methods described in Example 4 for either 10 or 30 minutes at -15 ° C. The control extraction was done with Methanol/Chloroform (9:1) at room temperature (RT).

Results are expressed in ppm (mg/kg dry Cannabis). Control extractions were carried out using a Methanol/Chloroform (9:1) procedure as in preceding examples.

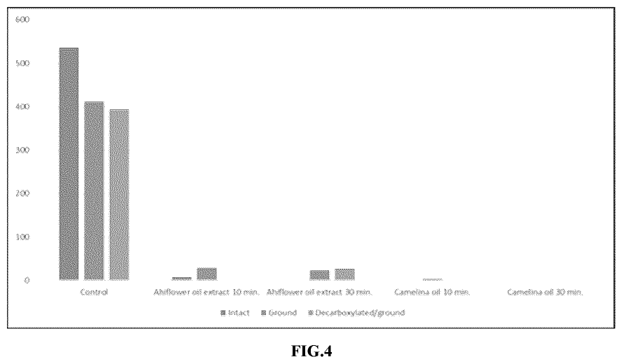

Extraction with cold lipid solvent greatly reduces chlorophyll extraction, as shown on FIG. 4. Results are expressed in ppm (mg/kg dry Cannabis).

What is claimed is:

1. A method of preparing a botanical extract comprising:
a. providing a plant material in an extraction chamber;
b. contacting a lipid solvent with the plant material;
c. extracting at least one bioactive molecule from the plant material into the lipid solvent for a period of time thereby producing a lipid solvent comprising a botanical extract;
d. filtering the lipid solvent comprising the botanical extract using a cold filtration/centrifugation system; and
e. recovering the lipid solvent from the filtration/centrifugation system thereby producing a botanical extract.

2. The method of claim 1, wherein contacting the lipid solvent with the plant material comprises releasing the lipid solvent from a solvent chamber into the extraction chamber.

3. The method of any one of claims 1-3, comprising heating the plant material prior to step (a).

4. The method of claim 3, wherein the plant material is heated to a temperature of about between 115° C. to 145° C.

5. The method of claim 3, wherein the plant material is heated to a temperature of about between 110° C. to 145° C.

6. The method of any one of claims 1-5, wherein the period of time is no more than 1 hour.

7. The method of any one of claims 1-5, wherein the period of time is between about 30 and about 60 minutes.

8. The method of any one of claims 1-5, wherein the period of time is between about 10 and about 30 minutes.

9. The method of any one of the preceding claims, further comprising agitating the contents of the extraction chamber during step (c).

10. The method of any one of the preceding claims, further comprising sonicating the Cannabis plant material and the lipid solvent prior to step (d).

11. The method of claim 10, wherein the sonication occurs during step (c).

12. The method of claim 10, wherein the sonication occurs before step (c).

13. The method of any one of the preceding claims, wherein the lipid solvent is at a temperature of about between 0° C. to −40° C.

14. The method of any one of the preceding claims, wherein step (c) is at about −5° C. to about −20° C., about −5° C. to about −10° C., or about 5° C. to about −15° C.

15. The method of any one of the preceding claims, wherein the lipid solvent comprises polyunsaturated fatty acids (PUFA).

16. The method of any one of the preceding claims, wherein the PUFA comprise omega-3 fatty acids.

17. The method of any one of the preceding claims, wherein the lipid solvent has a melting temperature below 0° C.

18. The method of any one of the preceding claims, wherein the lipid solvent has a melting temperature between about −8° C. to about −40° C.

19. The method of any one of the preceding claims, wherein the lipid solvent is selected from the group consisting fish oil, flax seed oil, camelina oil, evening primrose oil, black current oil, ahiflower seed oil, and a combination thereof.

20. The method of any one of the preceding claims, wherein the recovered lipid solvent from step (e) is returned to the extraction chamber and steps (b) through (e) are repeated.

21. The method of claim 20, wherein steps (b) through (e) are repeated 2×, 3×, 4×, 5×, 6×, 7×, 8×, 9×, or 10×.

22. The method of claim 20 or 21, wherein un-extracted plant material is added to the extraction chamber prior to repeating steps (c) through (e).

23. The method of any one of the preceding claims, wherein the botanical extract is bleached.

24. The method of any one of the preceding claims, wherein the botanical extract is subject to one or more additional purification methods.

25. The method of claim 24, wherein the one or more additional purification methods comprise molecular distillation or high-performance liquid chromatography (HPLC).

26. The method of any one of the preceding claims, wherein the plant material is fresh or dried.

27. The method of any one of the preceding claims, wherein the plant material is intact or milled.

28. The method of any one of the preceding claims, wherein the plant material is Cannabis.

29. The method of claim 28, wherein the Cannabis is Cannabis sativa, Cannabis indica or Cannabis ruderalis.

30. The method of claim 28 or 29, wherein the Cannabis is a hybrid.

31. The method of claim 28, wherein the Cannabis is industrial hemp.

32. The method of any one of claims claim 1-31, comprising winterization.

33. The method of any one of claims claim 1-32, comprising de-waxing.

34. The method of any one of the preceding claims, wherein the at least one bioactive molecule comprises a cannabinoid, a flavonoid or a terpene.

35. The method of claim 34, wherein the cannabinoid comprises Δ⁹-tetrahydrocannabinol (THC), cannabidiol (CBD), tetrahydrocannabinolic acid (THCA), cannabidiolic acid (CBDA), cannabigerolic acid (CBGA), cannabichromenenic acid (CBCA), cannabigerovarinic acid (CBGVA), tetrahydrocanabivarinic acid (THCVA), cannabidivarinic acid (CBDVA), cannabichromevarinic acid (CBCVA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabielsoin (CBE), cannabicitran (CBT), or a combination thereof.

36. The method of claim 34, wherein the cannabinoid comprises a combination of THC and CBD.

37. The method of claim 34, wherein the cannabinoid comprises a combination of THC, THCA, CBD and CBDA.

38. The method of any one of claims 34-36, wherein the terpene comprises myrcene, terpinolene, β-caryophyllene, selina-3 7(11)-diene, guaiol, 10-epi-y-eudesmol, β-eudesmol, α-eudesmol, bulnesol, α-bisabolol or a combination thereof.

39. A botanical extract produced by the method of any one of the preceding claims.

40. The botanical extract of claim 39, wherein the botanical extract is a resin.

41. The botanical extract of claim 39, wherein the botanical extract is a liquid.

42. A botanical extract, comprising at least one cannabinoid and a lipid solvent.

43. The botanical extract of claim 42, wherein the lipid solvent is selected from the group consisting of fish oil, flax seed oil, camelina oil, evening primrose oil, black current oil, ahiflower seed oil, and a combination thereof.

44. A composition comprising the botanical extract of any one of claims 39-43 and a pharmaceutically acceptable carrier, diluent or excipient.

45. The composition of claim 44, wherein the composition is formulated for oral administration.

46. The composition of claim 45, wherein the composition is formulated as a liquid, gel, softgel, powder, tablet, caplet, capsule, gelcap, food additive, drop, beverage, pill, lozenge, rinse, paste or gum.

47. The composition of claim 44, wherein the composition is formulated for topical administration.

48. The composition of claim 47, wherein the composition is formulated as a liquid, gel, cream, ointment, lotion, salve, balm or paste.

49. The composition of claim 44, wherein the composition is formulated for transmucosal administration, parenteral administration, subdermal administration, or inhalation.

50. The composition of claim 44, wherein the transmucosal administration comprises buccal administration or intra-nasal administration.

51. A method of making a Cannabis extract composition, comprising:
a. providing the botanical extract produced by the methods of any one of claims 1-38, and
b. mixing the botanical extract with a pharmaceutically acceptable carrier, diluent or excipient.

52. The method of claim 51, wherein the composition is formulated for oral administration.

53. The method of claim 52, wherein the composition is formulated as a liquid, gel, softgel, powder, tablet, caplet, capsule, gelcap, food additive, drop, beverage, pill, lozenge, rinse, paste or gum.

54. The method of claim 51, wherein the composition is formulated for topical administration.

55. The method of claim 54, wherein the composition is formulated as a liquid, gel, cream, ointment, lotion, salve, balm or paste.

56. The method of claim 51, wherein the composition is formulated for transmucosal administration, parenteral administration, subdermal administration, or inhalation.

57. The method of claim 56, wherein the transmucosal administration comprises buccal administration or intra-nasal administration.
